(19) United States
(12) Patent Application Publication
(10) Pub. No.: US 2026/0137452 A1
(43) Pub. Date: May 21, 2026

(54) MANUFACTURING METHOD FOR JAW FIXATION AND MORPHOLOGY FILLER

(71) Applicant: Sichuan University, Chengdu City (CN)

(72) Inventors: En Luo, Chengdu City (CN); Zhaokun Zhu, Chengdu City (CN); Liwei Huang, Chengdu City (CN); Yao Liu, Chengdu City (CN); Shibo Liu, Chengdu City (CN); Zhen Liu, Chengdu City (CN)

(21) Appl. No.: 19/055,905

(22) Filed: Feb. 18, 2025

(30) Foreign Application Priority Data
Nov. 15, 2024 (CN) 202411629196.0

(51) Int. Cl. A61B 34/10; A61B 17/00; A61B 17/15; A61B 17/56; A61F 2/00; A61F 2/28; A61F 2/30; A61F 2/46; B33Y 50/00; B33Y 80/00; G05B 19/4097

(52) U.S. Cl. CPC A61B 34/10; A61B 17/151; A61F 2/0095; B33Y 50/00; B33Y 80/00; G05B 19/4097; A61B 2017/00526; A61B 2017/568; A61B 2034/102; A61B 2034/108; A61F 2002/2807; A61F 2002/30948; A61F 2002/30952; A61F 2002/30985; A61F 2002/4633; G05B 2219/35134

(57) ABSTRACT

Provided is a manufacturing method for a jaw fixation and morphology filler, including the following steps: the primary design of a filler: designing a first PEEK filler according to imaging data of bone morphology of patients; design of a positioning guide plate: designing an osteotomy line and positioning holes on the positioning guide plate; the secondary design of the filler: simulating osteotomy to obtain postoperative new bone morphology, and subtracting a part of the first PEEK filler overlapped with a free bone block to obtain a second PEEK filler; the tertiary design of the filler: designing retention holes on the second PEEK filler, and performing edge trimming and surface smoothing treatment to obtain a third PEEK filler; and the manufacturing of the filler and the positioning guide plate: manufacturing the filler and the positioning guide plate according to the third PEEK filler and the positioning guide plate.

### CROSS-REFERENCE TO RELATED APPLICATION

This patent application claims the benefit and priority of Chinese Patent Application No. 202411629196.0 filed with the China National Intellectual Property Administration on Nov. 15, 2024, the disclosure of which is incorporated by reference herein in its entirety as part of the present application.

### TECHNICAL FIELD

The present disclosure relates to the technical field of surgery, and in particular to a manufacturing method for a jaw fixation and morphology filler.

### BACKGROUND

Osteotomy movement surgery of oral cavity and craniomaxillofacial region (such as maxillary osteotomy) is a common surgical method for treating the oral dento-maxillofacial and craniomaxillofacial deformities and the other diseases. After osteotomy movement surgery, the bone blocks are usually fixed with titanium plates and titanium nails. However, there is biocompatibility problems for titanium materials, which may cause adverse reactions such as metal allergy and imaging artifacts. Moreover, after osteotomy movement surgery, the patient may still have poor bone morphology in the fixed areas (such as nasal base and paranasal area, orbital region, and anterior wall area of maxilla.), which causes poor facial plumpness and affects the facial aesthetics. Therefore, an additional filling surgery is needed for patient, which causes increases in terms of operation time, complexity, and risk.

### SUMMARY

An objective of the present disclosure is to solve the technical problem above, and a manufacturing method for a jaw fixation and morphology filler is provided. The jaw fixation and morphology filler made of PEEK (polyether-ether-ketone) can be manufactured, which has excellent mechanical properties, biocompatibility and elastic modulus close to that of human bones, and can achieve the dual functions of bone block fixation and bone morphology filling after an osteotomy movement surgery. In the present disclosure, the surgical process is simplified, the postoperative aesthetic effect is improved, and there is no artifact in imaging, convenient for postoperative evaluation, with broad clinical application prospects.

In order to achieve the objective above, the present disclosure provides the following solutions: a manufacturing method for a jaw fixation and morphology filler includes the following steps:
primary design of a jaw fixation and morphology filler: collecting imaging data of bone morphology of an operation area of a patient, and designing a first PEEK filler according to the imaging data of the bone morphology;
design of a positioning guide plate: designing the positioning guide plate according to the imaging data of the bone morphology, and designing an osteotomy line and positioning holes on the positioning guide plate, where the osteotomy line is configured for guiding a bone saw to cut the operation area, and the positioning holes are configured for fixing the positioning guide plate at a bone surface of the operation area and leaving marking holes on the bone surface;
secondary design of the jaw fixation and morphology filler: simulating osteotomy according to a solution of an osteotomy movement surgery to acquire postoperative new bone morphology of the operation area, and subtracting a part of the first PEEK filler overlapped with a free bone block with the new bone morphology to obtain a design solution of a second PEEK filler;
tertiary design of the jaw fixation and morphology filler: designing retention holes on the second PEEK filler, enabling the retention holes to be in one-to-one correspondence with the marking holes on the bone surface, and carrying out edge trimming and surface smoothing treatment on the second PEEK filler according to the design solution of the second PEEK filler to obtain a design solution of a third PEEK filler; and
manufacturing of the jaw fixation and morphology filler and the positioning guide plate: manufacturing the jaw fixation and morphology filler and the positioning guide plate according to the design solutions of the third PEEK filler and the positioning guide plate.

Preferably, three-dimensional imaging data of the bone morphology of the operation area of the patient is obtained by CT (computed tomography) scanning.

Preferably, the three-dimensional imaging data is imported into a computer modeling software, and the positioning guide plate and the jaw fixation and morphology filler are designed by the computer modeling software.

In some embodiments, the computer modeling software adopts a combined use of FreeForm Modeling Plus software and CAD (Computer-aided design) software.

In some embodiments, the part of the first PEEK filler overlapped with the free bone block is subtracted by Boolean operation.

In some embodiments, the positioning guide plate and the third PEEK filler are manufactured by 3D printing according to model data output by the computer modeling software.

In some embodiments, after the positioning guide plate and the third PEEK filler are manufactured, the positioning guide plate and the third PEEK filler are disinfected and sterilized.

In some embodiments, the positioning guide plate and the third PEEK filler after disinfection and sterilization are packaged.

In some embodiments, the operation area is maxilla, or a chinbone region.

Compared with the prior art, the present disclosure has the following technical effects:
According to a manufacturing method for a jaw fixation and morphological filler, a jaw fixation and morphological filler made of PEEK can be manufactured, the Chinese name or full name of PEEK is polyether-ether-ketone, which has excellent mechanical properties and biocompatibility, and thus can not generate adverse reactions such as metal allergy and inflammation when used as fixing plates. Meanwhile, the PEEK with an elastic modulus close to that of human bones is excellent when made to fillers. Therefore, the PEEK can serve as both a fixation plate and a filler, and can complete the functions of bone block fixation and bone morphology filling in the same operation, and there is no need of another operation. In the present disclosure, the operation time, complexity and risk are reduced, the dysplasia of bone morphology is filled, the facial plumpness is improved, and the postoperative facial contour aesthetics of the patient is improved. Moreover, the characteristic that the elastic modulus of the PEEK is close to that of human bones can also reduce a phenomenon of stress shielding, and there is no metal artifact in the imaging examination, convenient for postoperative evaluation, with important clinical significance.

### BRIEF DESCRIPTION OF THE DRAWINGS

To describe the technical solutions in the embodiments of the present disclosure or in the prior art more clearly, the following briefly introduces the accompanying drawings required in the embodiments. Apparently, the accompanying drawings in the following description show merely some embodiments of the present disclosure, and those skilled in the art may still derive other drawings from these accompanying drawings without creative efforts.

FIG. 1 is a simulation diagram of a positional relationship between maxilla and a first PEEK filler according to an embodiment of the present disclosure;

FIG. 2 is a front simulation view of a morphological structure of a first PEEK filler for maxilla according to an embodiment of the present disclosure;

FIG. 3 is a side simulation view of a morphological structure of a first PEEK filler for maxilla according to an embodiment of the present disclosure;

FIG. 4 is a rear simulation view of a morphological structure of a first PEEK filler for maxilla according to an embodiment of the present disclosure;

FIG. 5 is a simulation diagram of a positional relationship between maxilla and a positioning guide plate according to an embodiment of the present disclosure;

FIG. 6 is a front simulation view of a morphological structure of a positioning guide plate according to an embodiment of the present disclosure;

FIG. 7 is a side simulation view of a morphological structure of a positioning guide plate according to an embodiment of the present disclosure;

FIG. 8 is a rear simulation view of a morphological structure of a positioning guide plate according to an embodiment of the present disclosure;

FIG. 9 is a simulation diagram of a positional relationship between maxilla and a second PEEK filler according to an embodiment of the present disclosure;

FIG. 10 is a front simulation view of a morphological structure of a second PEEK filler for maxilla according to an embodiment of the present disclosure;

FIG. 11 is a side simulation view of a morphological structure of a second PEEK filler for maxilla according to an embodiment of the present disclosure;

FIG. 12 is a rear simulation view of a morphological structure of a second PEEK filler for maxilla according to an embodiment of the present disclosure;

FIG. 13 is a simulation diagram of a positional relationship between maxilla and a third PEEK filler according to an embodiment of the present disclosure;

FIG. 14 is a front simulation view of a morphological structure of a third PEEK filler for maxilla according to an embodiment of the present disclosure;

FIG. 15 is a side simulation view of a morphological structure of a third PEEK filler for maxilla according to an embodiment of the present disclosure;

FIG. 16 is a rear simulation view of a morphological structure of a third PEEK filler for maxilla according to an embodiment of the present disclosure;

FIG. 17 is a left simulation view of maxilla and a third PEEK filler after fixation according to an embodiment of the present disclosure;

FIG. 18 is a front simulation view of maxilla and a third PEEK filler after fixation according to an embodiment of the present disclosure;

FIG. 19 is a right simulation view of maxilla and a third PEEK filler after fixation according to an embodiment of the present disclosure;

FIG. 20 is a rear simulation view of maxilla and a third PEEK filler after fixation according to an embodiment of the present disclosure;

FIG. 21 is a simulation diagram of a positional relationship between a chinbone region and a third PEEK filler according to an embodiment of the present disclosure;

FIG. 22 is a front simulation view of a morphological structure of a third PEEK filler for a chinbone region according to an embodiment of the present disclosure;

FIG. 23 is a side simulation view of a morphological structure of a third PEEK filler for a chinbone region according to an embodiment of the present disclosure;

FIG. 24 is a rear simulation view of a morphological structure of a third PEEK filler for a chinbone region according to an embodiment of the present disclosure.

Reference numerals in the drawings: 1—positioning guide plate; 2—first PEEK filler; 3—second PEEK filler; 4—third PEEK filler; 5—osteotomy line; 6—positioning hole; 7—retention hole; 8—screw; 9—maxilla; 10—chinbone region.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

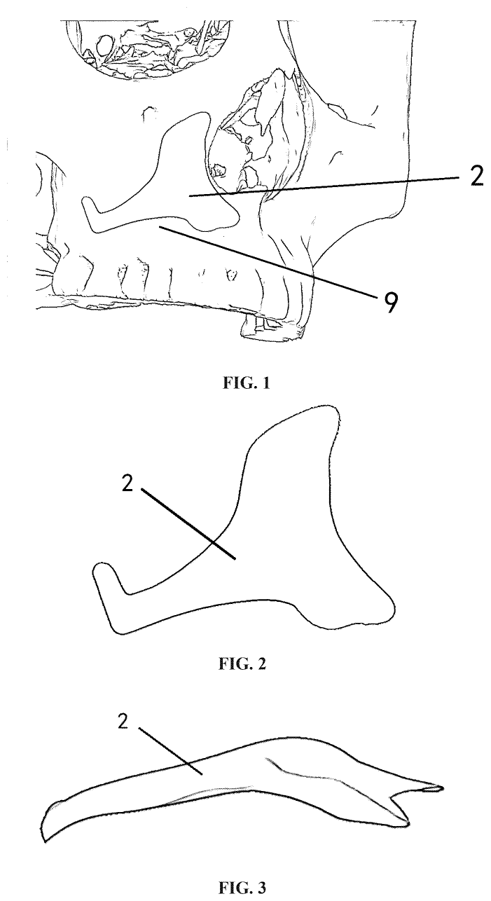

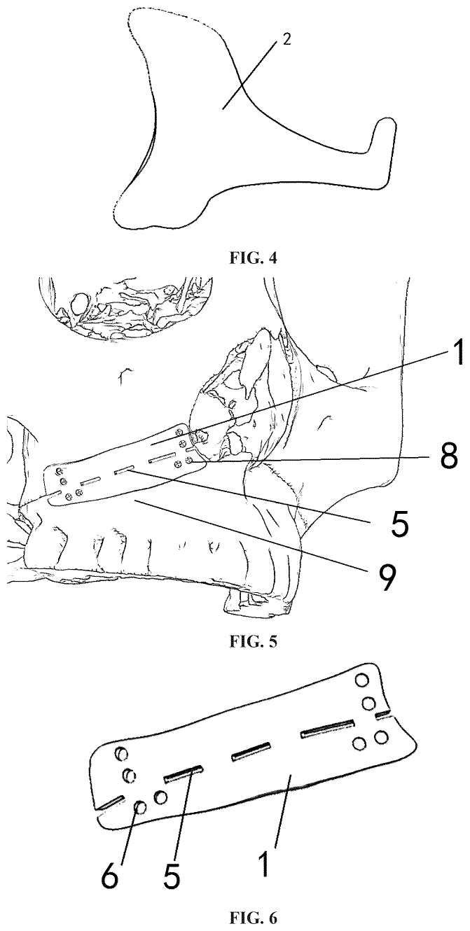

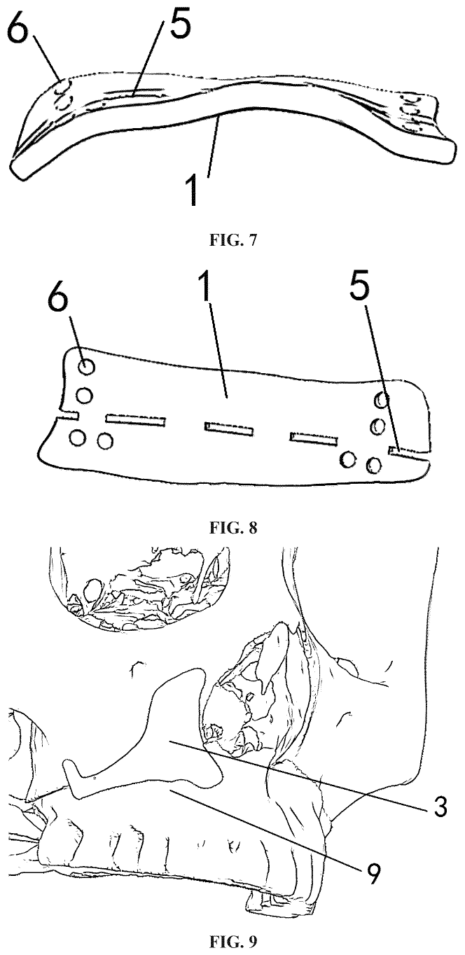

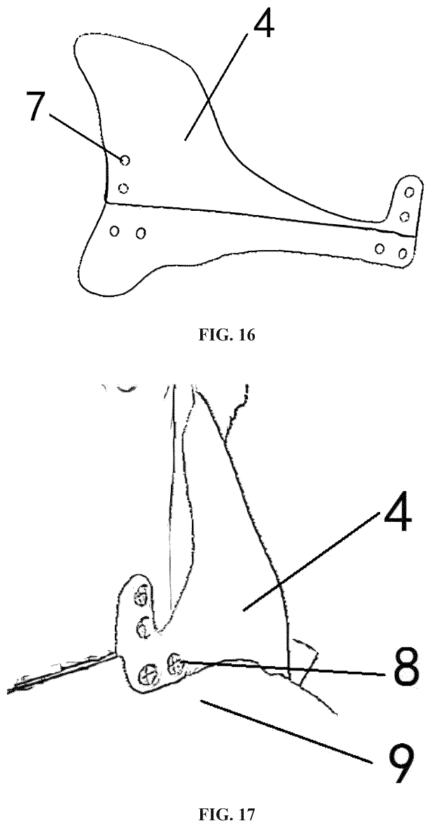

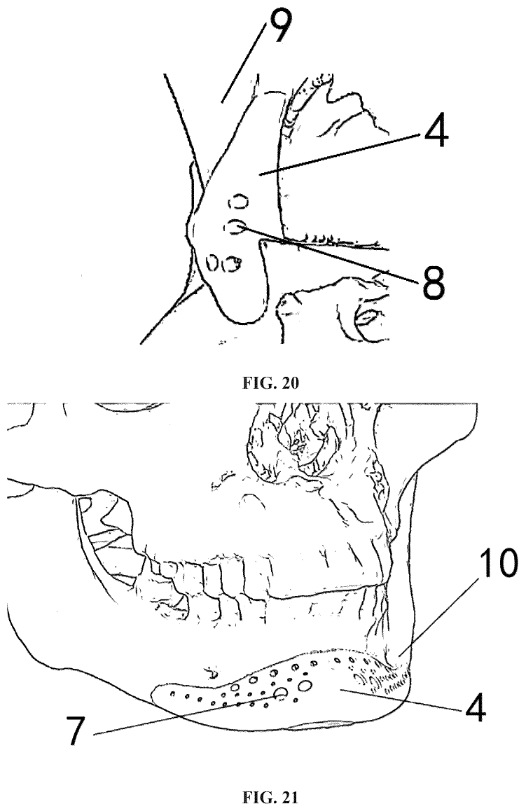

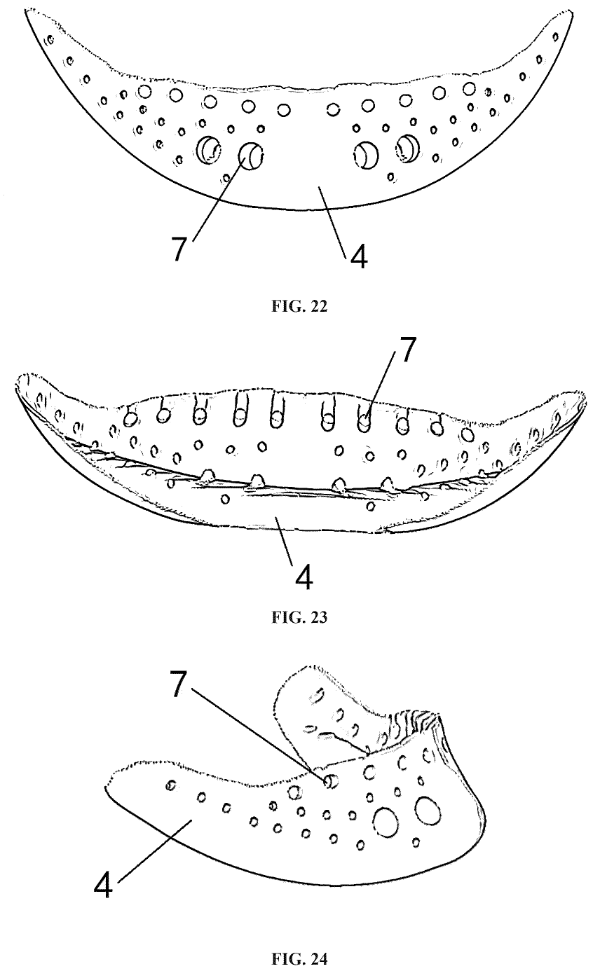

This embodiment provides a manufacturing method for a jaw fixation and morphology filler, as shown in FIG. 1 to FIG. 24, including the following steps:
the primary design of a jaw fixation and morphology filler: imaging data of bone morphology of an operation area (such as maxilla 9, or a chinbone region 10) of a patient is collected, and a first PEEK filler 2 is designed according to the imaging data of the bone morphology;
the design of a positioning guide plate: a positioning guide plate 1 is designed according to the imaging data of the bone morphology, and an osteotomy line 5 and positioning holes 6 are designed on the positioning guide plate 1, where the osteotomy line 5 is used for guiding a bone saw to cut the operation area, and the positioning holes 6 are used for fixing the positioning guide plate 1 at a bone surface of the operation area and leaving marking holes on the bone surface;
the secondary design of the jaw fixation and morphology filler: osteotomy is simulated according to a solution of an osteotomy movement surgery to acquire postoperative new bone morphology of the operation area, and subtracting a part of the first PEEK filler 2 overlapped with a free bone block with the new bone morphology to obtain a design solution of a second PEEK filler 3;
the tertiary design of the jaw fixation and morphology filler: retention holes 7 are designed on the second PEEK filler 3, and the retention holes 7 are in one-to-one correspondence with the marking holes on the bone surface after the simulated osteotomy movement surgery. The edge trimming and surface smoothing treatment on the second PEEK filler 3 are carried out according to the design solution of the second PEEK filler 3, thus obtaining the design solution of a third PEEK filler 4; and
the manufacturing of the jaw fixation and morphology filler, as well as the positioning guide plate: the jaw fixation and morphology filler, as well as the positioning guide plate 1 are manufactured according to the design solutions of the third PEEK filler 4 and the positioning guide plate 1.

The present disclosure has the beneficial effects that:
According to a manufacturing method for a jaw fixation and morphological filler, a jaw fixation and morphological filler made of PEEK can be manufactured. the Chinese name or full name of PEEK is polyether-ether-ketone, which has excellent mechanical properties and biocompatibility, and thus can not generate adverse reactions such as metal allergy and inflammation when used as fixing plates. Meanwhile, the PEEK with an elastic modulus close to that of human bones is excellent when made to fillers. Therefore, the PEEK can serve as both a fixation plate and a filler, and can complete the functions of bone block fixation and bone morphology filling in the same operation, and there is no need of another operation. In the present disclosure, the operation time, complexity and risk are reduced, the dysplasia of bone morphology is filled, the facial plumpness is improved, and the postoperative facial contour aesthetics of the patient is improved. Moreover, the characteristic that the elastic modulus of the PEEK is close to that of human bones can also reduce a phenomenon of stress shielding, and there is no metal artifact in the imaging examination, convenient for postoperative evaluation, with important clinical significance.

The treatment process is as follows.
(1) Preoperative preparation:
   According to the surgical planning, the operator prepares the positioning guide plate 1 and the third PEEK filler 4 which are disinfected and sterilized.
(2) Surgical implementation:
   The positioning guide plate 1 is placed at a preset position of the operation area, marking holes are left on the bone surface through the positioning holes 6, and the operation area is cut with a bone saw along the osteotomy line 5, such that the free bone block at the operation area after osteotomy can easily move to the preset position.
(3) Use of the third PEEK:
   The retention holes 7 on the third PEEK filler 4 are aligned with the marking holes left on the bone surface, and then the third PEEK filler 4 is fixed through screws 8 to achieve bone block fixation and bone morphology filling.
(4) Postoperative treatment: the stability of the fixed bone block and the bone morphology filling effect are checked, and routine postoperative care and follow-up are carried out.

In an embodiment, as shown in FIG. 1 to FIG. 24, three-dimensional imaging data of the bone morphology is obtained by CT scanning of the operation area of the patient.

In an embodiment, as shown in FIG. 1 to FIG. 24, the three-dimensional imaging data is imported into computer modeling software, and the positioning guide plate and the jaw fixation and morphology filler are designed by the computer modeling software.

In an embodiment, as shown in FIG. 1 to FIG. 24, the computer modeling software adopts the combined use of FreeForm Modeling Plus software and CAD software.

In an embodiment, as shown in FIG. 1 to FIG. 24, the part of the first PEEK filler 2 overlapped with the free bone block is subtracted by Boolean operation.

In an embodiment, as shown in FIG. 1 to FIG. 24, the positioning guide plate 1 and the third PEEK filler 4 are manufactured by 3D printing according to the model data output by the computer modeling software.

In an embodiment, as shown in FIG. 1 to FIG. 24, after the positioning guide plate 1 and the third PEEK filler 4 are manufactured, the positioning guide plate 1 and the third PEEK filler 4 are disinfected and sterilized.

In an embodiment, as shown in FIG. 1 to FIG. 24, the positioning guide plate 1 and the third PEEK filler 4 after disinfection and sterilization are packaged.

In an embodiment, as shown in FIG. 1 to FIG. 24, the operation area is maxilla 9, and the osteotomy movement surgery is the osteotomy movement surgery for the maxillary.

At first, three-dimensional CT imaging data of bone morphology of the maxilla 9 of the patient is collected, and then imported into the FreeForm Modeling Plus software and CAD software for modeling. A first PEEK filler 2 is designed according to the modeling (as shown in FIG. 1 to FIG. 4).

Then, a positioning guide plate 1 is designed according to the modeling, and an osteotomy line 5 and positioning holes 6 are designed on the positioning guide plate 1, where the osteotomy line 5 is used for guiding a bone saw to cut the maxilla 9, and the positioning holes 6 are used for fixing the positioning guide plate 1 to a bone surface of the maxilla 9 and leaving marking holes on the bone surface (as shown in FIG. 5 to FIG. 8).

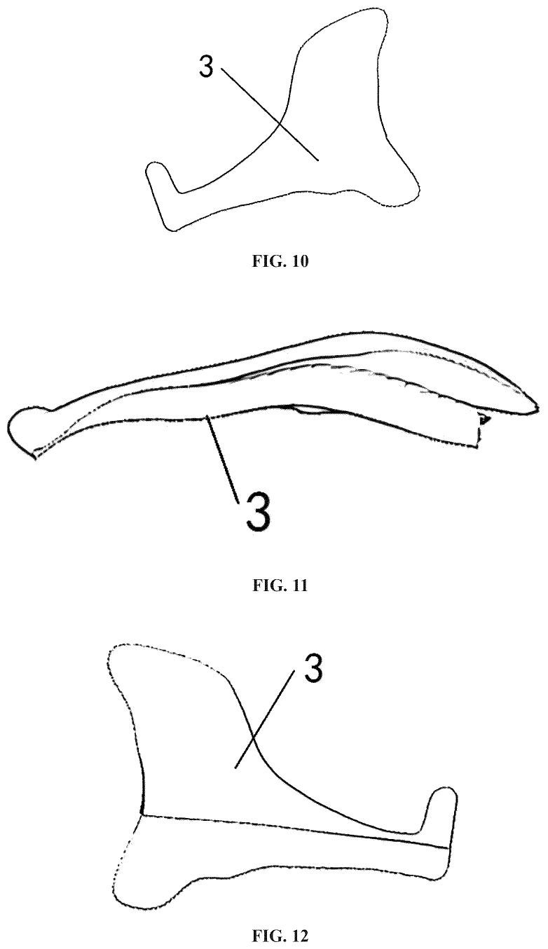

Then, according to the solution of the maxilla osteotomy movement surgery, the maxilla osteotomy movement surgery is simulated. An example of maxillary Le Fort I osteotomy advancement surgery is described. The free bone block of maxilla is advanced to obtain the movement amount of the free bone block of maxilla, and the part of the first PEEK filler 2 overlapped with the free bone block at maxilla is subtracted by Boolean operation to obtain the design solution of a second PEEK filler 3 (as shown in FIG. 9 to FIG. 12).

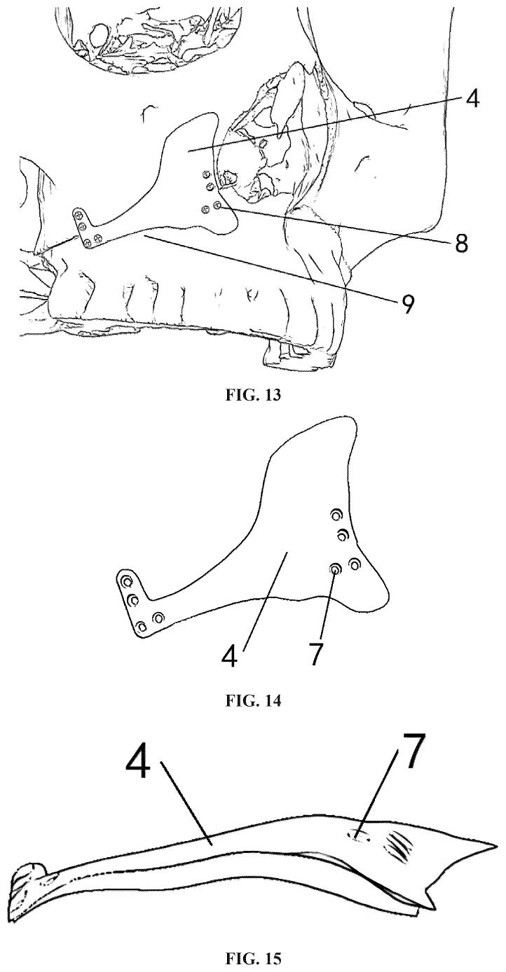

Then, retention holes 7 are designed on the second PEEK filler 3, and the retention holes 7 are in one-to-one correspondence with the marking holes on the bone surface of maxilla. The edge trimming and surface smoothing treatment of the second PEEK filler 3 are carried out, thus obtain the design solution for a third PEEK filler 4 (as shown in FIG. 13 to FIG. 16).

Then, 3D printing is carried out according to the design solution of the third PEEK filler 4 and the design solution of the positioning guide plate 1.

Finally, the third PEEK filler 4 and the positioning guide plate 1 are disinfected, sterilized, and packaged.

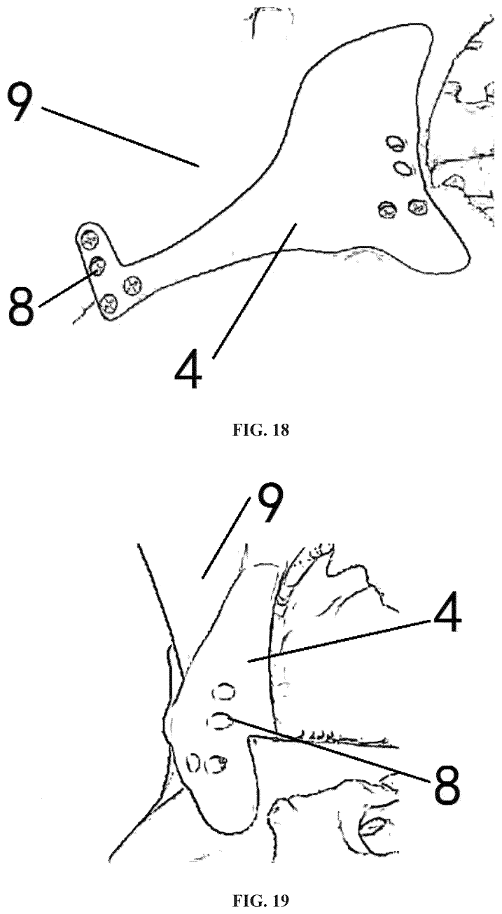

During operation, at first, the positioning guide plate 1 is used to complete the cut of the bone block and the mark of screw holes, and then the third PEEK filler 4 is used to complete the bone block fixation and bone morphology filling, and the fitting between the third PEEK filler 4 and the postoperative bone block is checked (as shown in FIG. 17 to FIG. 20).

In an embodiment, as shown in FIG. 1 to FIG. 24, the operation area is a chinbone region 10, and the osteotomy movement surgery is the osteotomy movement surgery for the chinbone: the design solution of manufacturing the third PEEK filler 4 is as shown in FIG. 21 to FIG. 24. The chinbone region 10 is a part of the mandible.

Specific examples are used herein for illustration of the principles and embodiments of the present disclosure. The description of the embodiments is merely used to help illustrate the method and its core principles of the present disclosure. In addition, those skilled in the art can make various modifications in terms of specific embodiments and application scope in accordance with the principles of the present disclosure. In conclusion, the content of this specification shall not be construed as a limitation to the present disclosure.

What is claimed is:

1. A manufacturing method for a jaw fixation and morphology filler, wherein the method comprises the following steps:
primary design of the jaw fixation and morphology filler: collecting imaging data of bone morphology of an operation area of a patient, and designing a first PEEK (polyether-ether-ketone) filler according to the imaging data of the bone morphology;
design of a positioning guide plate: designing the positioning guide plate according to the imaging data of the bone morphology, and designing an osteotomy line and positioning holes on the positioning guide plate, wherein the osteotomy line is configured for guiding a bone saw to cut the operation area, and the positioning holes are configured for fixing the positioning guide plate to a bone surface of the operation area and leaving marking holes on the bone surface;
secondary design of the jaw fixation and morphology filler: simulating osteotomy according to a solution of an osteotomy movement surgery to acquire postoperative new bone morphology of the operation area, and subtracting a part of the first PEEK filler overlapped with a free bone block with the new bone morphology to obtain a design solution of a second PEEK filler;
tertiary design of the jaw fixation and morphology filler: designing retention holes on the second PEEK filler, enabling the retention holes to be in one-to-one correspondence with the marking holes on the bone surface, and carrying out edge trimming and surface smoothing treatment on the second PEEK filler according to the design solution of the second PEEK filler to obtain a design solution of a third PEEK filler; and
manufacturing of the jaw fixation and morphology filler and the positioning guide plate: manufacturing the jaw fixation and morphology filler and the positioning guide plate according to the design solutions of the third PEEK filler and the positioning guide plate.

2. The manufacturing method for a jaw fixation and morphology filler according to claim 1, wherein three-dimensional imaging data of the bone morphology of the operation area of the patient is obtained by CT (computed tomography) scanning.

3. The manufacturing method for a jaw fixation and morphology filler according to claim 2, wherein the three-dimensional imaging data is imported into a computer modeling software, and the positioning guide plate and the jaw fixation and morphology filler are designed by the computer modeling software.

4. The manufacturing method for a jaw fixation and morphology filler according to claim 3, wherein the computer modeling software adopts a combined use of FreeForm Modeling Plus software and CAD (Computer-aided design) software.

5. The manufacturing method for a jaw fixation and morphology filler according to claim 4, wherein the part of the first PEEK filler overlapped with the free bone block is subtracted by Boolean operation.

6. The manufacturing method for a jaw fixation and morphology filler according to claim 3, wherein the positioning guide plate and the third PEEK filler are manufactured by 3D printing according to model data output by the computer modeling software.

7. The manufacturing method for a jaw fixation and morphology filler according to claim 1, wherein after the positioning guide plate and the third PEEK filler are manufactured, the positioning guide plate and the third PEEK filler are disinfected and sterilized.

8. The manufacturing method for a jaw fixation and morphology filler according to claim 7, wherein the positioning guide plate and the third PEEK filler after disinfection and sterilization are packaged.

9. The manufacturing method for a jaw fixation and morphology filler according to claim 1, wherein the operation area is maxilla, and the osteotomy movement surgery is maxillary osteotomy movement surgery.

10. The manufacturing method for a jaw fixation and morphology filler according to claim 1, wherein the operation area is a chinbone region, and the osteotomy movement surgery is chinbone osteotomy movement surgery.

11. The manufacturing method for a jaw fixation and morphology filler according to claim 9, wherein three-dimensional imaging data of the bone morphology of the operation area of the patient is obtained by CT (computed tomography) scanning.

12. The manufacturing method for a jaw fixation and morphology filler according to claim 11, wherein the three-dimensional imaging data is imported into a computer modeling software, and the positioning guide plate and the jaw fixation and morphology filler are designed by the computer modeling software.

13. The manufacturing method for a jaw fixation and morphology filler according to claim 12, wherein the computer modeling software adopts a combined use of FreeForm Modeling Plus software and CAD (Computer-aided design) software.

14. The manufacturing method for a jaw fixation and morphology filler according to claim 13, wherein the part of the first PEEK filler overlapped with the free bone block is subtracted by Boolean operation.

15. The manufacturing method for a jaw fixation and morphology filler according to claim 12, wherein the positioning guide plate and the third PEEK filler are manufactured by 3D printing according to model data output by the computer modeling software.

16. The manufacturing method for a jaw fixation and morphology filler according to claim 9, wherein after the positioning guide plate and the third PEEK filler are manufactured, the positioning guide plate and the third PEEK filler are disinfected and sterilized.

17. The manufacturing method for a jaw fixation and morphology filler according to claim 16, wherein the positioning guide plate and the third PEEK filler after disinfection and sterilization are packaged.

18. The manufacturing method for a jaw fixation and morphology filler according to claim 10, wherein three-dimensional imaging data of the bone morphology of the operation area of the patient is obtained by CT (computed tomography) scanning.

19. The manufacturing method for a jaw fixation and morphology filler according to claim 18, wherein the three-dimensional imaging data is imported into a computer modeling software, and the positioning guide plate and the jaw fixation and morphology filler are designed by the computer modeling software.

20. The manufacturing method for a jaw fixation and morphology filler according to claim 19, wherein the computer modeling software adopts a combined use of FreeForm Modeling Plus software and CAD (Computer-aided design) software.
